(19) United States
(12) Patent Application Publication
(10) Pub. No.: US 2003/0104483 A1
(43) Pub. Date: Jun. 5, 2003

(54) Liquid chromatography/fourier-transform mass spectrometry/electron capture dissociation for the analysis of proteins

(75) Inventors: Walter Carroll Davidson, Croton, NY (US); Lee Frego, Hopewell Junction, NY (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT

(21) Appl. No.: 10/301,110

(22) Filed: Nov. 21, 2002

Related U.S. Application Data
(60) Provisional application No. 60/334,381, filed on Nov. 30, 2001.

(51) Int. Cl. G01N 33/53; C12Q 1/37; G01N 33/00

(52) U.S. Cl. 435/7.1; 436/86; 435/23

(57) ABSTRACT

ECD (Electron Capture Dissociation) FTMS (Fourier-Transform Mass Spectrometry) induced fragmentation is employed to generate sequence information for a protein enzymatic digest. The digest is initially separated by liquid chromatography, e.g., reversed phase μHPLC, and then ionized “on-line”. The ions thus formed may be accumulated in the interface hexapole prior to injection and trapping in the FTMS cell. Typically, no parent ion isolation is performed. The trapped ions are subjected to a pulse of electrons to induce fragmentation. Broad band spectra are acquired continuously to produce a three-dimensional LC/MS data set. The spectra are dominated by c and to a lesser degree z ions, which provide nearly complete sequence coverage. External calibration provides good mass accuracy and resolution, typical of FTMS. Thus, LC/ECD-FTMS is shown to be a highly informative method for the analysis of enzymatic protein digests.

### RELATED APPLICATIONS

Benefit of U.S. Provisional Application Serial No. 60/334,381, filed on November 30, 2001 is hereby claimed.

### BACKGROUND OF THE INVENTION

LC/MS (liquid chromatography/mass spectrometry) analysis of protein enzymatic digests is an important technology with a wide variety of applications including protein sequencing, analysis of post-translational modifications, proteomics, quality control of therapeutic and other protein preparations, etc. Conventional methods usually involve electrospray (ESI) ionization of the effluent from a reversed phase HPLC separation followed by mass spectrometry with any of a variety of fragmentation techniques. Fragmentation caused by collisions within the ESI interface is often employed to generate sequence information [1]. Tandem MS/MS using triple quadrupole or quadrupole time of flight instruments is often the method of choice for such analyses [2,3,4]. Ions of interest, usually protonated molecular ions, are isolated in the first step, fragmented by collisional activation in a multipolar rf collision cell and analyzed by a second mass spectrometer. The techniques are extremely useful. The spectra produced do, however, have some limitations. Often incomplete sequence coverage is obtained, internal fragmentation may complicate interpretation, [5] and often information relating to post-translational modification is lost due to ejection of the modification prior to backbone cleavage. MS/MS experiments amenable to FTMS (Fourier-Transform Mass Spectrometry), such as Sustained Off-Resonance Ionization (SORI) performed by addition of a collision gas to excited ions in a FTMS cell, suffer similar limitations [6].

The recent introduction of ECD-FTMS (Electron Capture Dissociation-Fourier-Transform Mass Spectrometry) provides a fragmentation technique that avoids many of these limitations. The mechanistic aspects of this technique have been studied and discussed by its originators and others [7,8,9,10]. It typically induces far more universal backbone cleavage and produces few internal fragments. It produces primarily c and z ions via cleavage at the Cα-N bond. The method has been applied principally to obtaining sequence information for intact proteins [11,12] and analysis of isolated peptides with post-translational modifications such as glycosylation [13] or phosphorylation [14,15]. The ECD-FTMS spectra of 5 synthetic peptides, introduced by direct infusion, have been reported along with a comparison to the more common SORI-FTMS/MS of these peptides [16]. That report observed much more complete sequence coverage via ECD. The present invention covers a novel application of ECD—the use ECD for a comprehensive, on-line analysis of a protein digest by LC/MS.

Pepsin cleavage takes place at low pH and it is less predictable than other commonly employed enzymes [17]. These often-troublesome properties were found to be useful in the present inventive method. Peptides with a range of polarities and terminal groups were generated from a single digest to rapidly gain an indication of the scope of this method. The present method was initially developed in order to study the applicability of this fragmentation technique to aid D₂O exchange studies wherein the rapidity and low pH optimum of pepsin are essential [18]. This also suggested the use of cytochrome c as a substrate since it has been extensively studied by D₂O exchange MS [19]. It furthermore gave an additional motivation for the development of a novel technique that does not require a parent isolation step, as the parent ions in such studies “shift” with the extent of exchange. Although the present inventive method was developed initially with the aforementioned limited purposes in mind, it will be appreciated by those skilled in the art that the present method as hereinafter described has broad applicability in the analysis of proteins and their enzymatic digests.

### SUMMARY OF THE INVENTION

The present invention is directed to a process for analyzing a protein, comprising:

(a) digesting a protein with an enzyme to produce a protein digest;

(b) subjecting the protein digest produced in step (a) to liquid chromatography to separate the protein digest into components;

(c) ionizing the protein digest components produced in step (b) to produce multiply charged ions;

(d) trapping the ions produced in step (c) in an analysis cell of a fourier transform mass spectrometer;

(e) irradiating the trapped ions of step (d) with electrons to produce fragment ions; and

(f) obtaining mass spectral data with respect to the fragment ions produced in step (e) to characterize one or more components of the protein digest.

It would be understood by a person skilled in the art that a number of different types of enzymes may be employed in step (a) for protein digestion. In one embodiment, the enzyme used is selected from pepsin, trypsin, chymotrypsin or Endo Lys C.

In one embodiment, the liquid chromatography used in step (b) is high pressure liquid chromatography (HPLC), e.g., reversed phase HPLC. In a preferred embodiment, the protein digest components obtained upon subjecting the protein digest to liquid chromatography are introduced directly into the ion source of a mass spectrometer, e.g., a FTMS, for the subsequent ionization step. That is, in this embodiment there is no intermediate step, such as a further physical separation or other analysis step, between the liquid chromatography and ionization steps of the process.

In another embodiment, the ionizing of the protein digest components in step (c) is conducted using electrospray ionization (ESI), e.g., using the ion source of a suitable mass spectrometer (MS or FTMS). It shall be understood, however, that the present invention broadly covers any method that has been used, is presently being used or may in the future be used ionize the protein digest components.

In another embodiment, a metal filament is used in step (e) to produce the electrons that are used to irradiate the trapped ions to produce fragment ions (i.e., ionic protein fragments). This is an application of the Electron Capture Dissociation technique in the present inventive method. It shall be understood, however, that the present invention covers any method that has been used, is presently being used or may in the future be used to irradiate the trapped ions with electrons to produce the fragment ions.

A parent ion isolation step is not necessary in the present inventive method, although if it is employed it is generally conducted after trapping the ions in the FTMS cell and prior to the electron irradiation step. Accordingly, in one embodiment there is no parent ion isolation during the process; and in another embodiment step (d) (ion trapping step) is followed by one or more parent ion isolation steps prior to the irradiation of the trapped ions in step (e). The parent ion isolation step is generally used when it is desirable to enhance the information content of the subsequently produced fragment ions.

In yet another embodiment, the ions are produced and isolated in step (c) using a mass spectrometer other than a fourier transform mass spectrometer, and these ions are then admitted into a fourier transform mass spectrometer for trapping in step (d). In this way, parent ion isolation can be conducted using a separate mass spectrometer (other than the FTMS).

As will be appreciated, the mass spectral data obtained with respect to the fragment ions may be used to characterize one or more components of the protein digest to assist in the analysis of the protein structure. For example, the spectral data may be analyzed to elucidate or validate the secondary structure of the protein or a mixture of proteins. This analysis can be conducted by manual inspection of the data, or by such manual inspection assisted by software data interpretation techniques known in the art.

It will also be appreciated that in one embodiment the process is run continuously in order to obtain multiple spectra with respect to the fragment ions that are produced in order to enhance the final information content.

### BRIEF DESCRIPTION OF THE DRAWINGS

FIGS. 1A to 1F: Extracted ion chromatograms of [M+2H^{]+2} obtained via μHPLC/ECD-FTMS for several peptides which span the cytochrome c sequence from amino acid residues 22-104.

FIGS. 2A to 2D: Spectra of peptide 81-94 (IFAGIKKKTEREDL) of cytochrome c obtained via various fragmentation techniques:

FIG. 2A: Shows the μHPLC/ECD/FTMS spectrum without parent ion isolation. Note that mass assignments for the indicated N-terminal ions were accurate to better than 4ppm within the calibration range (up to m/z=1348) for this peptide.

FIG. 2B: Shows the spectrum for μHPLC/ECD/FTMS/MS using parent ion isolation of [M+3H]⁺³.

FIG. 2C: Shows the spectrum produced via μHPLC/FTMS with in-source cone skimmer fragmentation.

FIG. 2D: Shows the spectrum for LC/MS/MS using a triple quadrupole instrument with CAD.

### DETAILED DESCRIPTION OF THE INVENTION

As would be appreciated by a person skilled in the art, the process of the present invention is a novel combination of analytical procedures hereinbefore themselves individually known in the art. Accordingly, since LC, MS, ESI and FTMS/ECD are themselves individually known procedures in the analysis of proteins, a person skilled in the art could practice the present inventive method using the guidance provided by the present disclosure together with the knowledge in the art.

As will also be appreciated from the results presented hereinafter, the novel combination of analytical procedures employed in the present inventive method produces unexpectedly superior analytical results as compared to conventional methods in the art for the analysis of protein digests. Thus, the present method is superior to conventional methods for the analysis of proteins.

In order for this invention to be more fully understood, the following examples are set forth. These examples are for the purpose of illustrating embodiments of this invention, and are not to be construed as limiting the scope of the invention in any way. The examples which follow are illustrative and, as recognized by one skilled in the art, particular equipment, materials, reagents, processing parameters and conditions could be modified as needed to obtain optimal results in any particular application of the present inventive method.

### EXAMPLE 1

1. Summary

ECD (Electron Capture Dissociation) FTMS (Fourier-transform mass spectrometry) induced fragmentation employed to generate sequence information for a pepsin digest of cytochrome c is described. The digest was separated by reversed phase μHPLC and ionized “on-line” by electrospray ionization. The ions thus formed were accumulated in the interface hexapole prior to injection and trapping in the FTMS cell. Typically, no parent ion isolation was performed. The trapped ions were subjected to a pulse of electrons to induce fragmentation. Broad band spectra were acquired continuously to produce a three-dimensional LC/MS data set. The spectra were dominated by c and to a lesser degree z ions, which provided nearly complete sequence coverage. External calibration provided good mass accuracy and resolution, typical of FTMS. Thus μHPLC/ECD-FTMS is shown to be a highly informative method for the analysis of enzymatic protein digests.

2. Experimental

A Bruker (Billerica, Mass.) Apex II FTMS with 7.0 Tesla shielded magnet and ESI interface was employed. The cell contains a metal filament used to generate electrons. The supplied Bruker pulse sequence for ECD was employed. Except as noted, the parent ion isolation step was removed. The sequence was modified to allow acquisition of multiple spectra. Thus the data acquisition was performed with no modification to the supplied hardware and minor modification of the supplied pulse sequence. Ions were accumulated for 0.5 seconds before gated trapping in the FTMS cell; no cooling gas was employed. The trapped ions were irradiated for 30 milliseconds with electrons produced by a metal filament operated at 6V and 3.26 μA. 6 such spectra were accumulated to produce each stored 256K spectrum with detection from m/z 292 to 2000. After a 12 minute delay time, a total of 128 spectra were acquired over 40 minutes. The external calibration was made by assignment of ECD fragment ions for substance P. Thus calibration is extrapolated above m/z 1347. The spectra were apodized and transformed after the analysis. They were converted to Micromass MassLynx format (Sierra Analytics, Calif.) and reviewed by use of Mass Lynx software (Micromass, Manchester UK) software.

A Quatro Ultima triple quadrupole MS (Micromass, Manchester, UK) was employed to obtain LC/MS/MS data. Source temperature was 120° C. and desolvation temperature was 150° C. Cone voltage was set at 50V and capillary voltage was 4 kV. Q1 was set to isolate mass 824.5 amu and argon was used as the collision gas. Collision voltage was 30V. Q3 was scanned from 200-2100 with a 3-second scan time.

50 ug of cytochrome c (in 100 mM NaH2PO4, pH 2.5) was digested with pepsin (50 ug) at 0° C. for 5 minutes. The entire digest (60 ul) was injected into a Peptide Trap (Michrom Bioresources Auburn, Calif.) contained in the injection loop. μHPLC was performed with a SCL 10A liquid chromatograph (Shimadzu, Colombia, Md.). Flow rate was 600 μl/min split 100:1 before the injector by a #AC-70 splitter (LC Packings). A Pepmap C18 150×0.32 mm column (LC Packings, San Francisco, Calif.) was employed. The A mobile phase was 99, 1, 0.1 water, acetonitrile, formic acid; B mobile phase was 5, 95, 0.1 water, acetonitrile, formic acid. The gradient was programmed to 60% B at 28 min and 100% B at 38 min. Acquisition was started at 12 min. This HPLC system and these conditions were used for the both ECD experiments and the triple quad MS/MS analysis.

3. Results

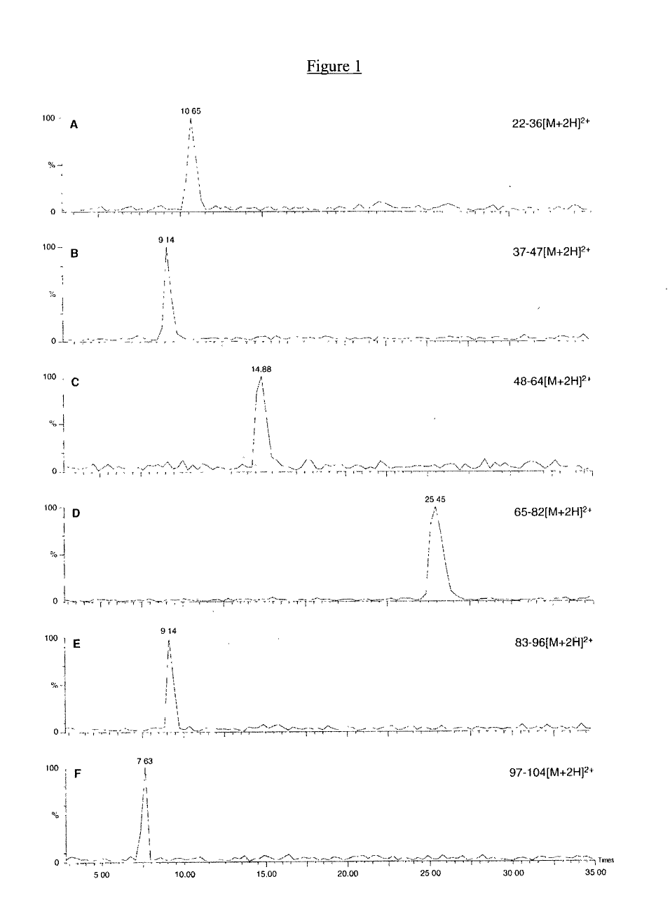

The enzymatic digest was separated by μHPLC and analyzed on line by broad band ECD-FTMS as detailed above. FIG. 1 shows extracted mass chromatograms for the [M+2H]²⁺ molecular ions of several peptides. These were chosen to show complete coverage of the cytochrome c sequence from residues 22-104. They demonstrate the chromatographic separation obtained and show excellent signal to noise. The overall results of this experiment are summarized in Table 1. Very good sequence coverage was obtained. The residues 1-22 gave weak or undetectable peptides. These residues contain a cyclic covalent modification by the heme function and are thus atypical of the peptides in mixtures produced by enzymatic digestion. They also give weak or undetectable response when analyzed by LC/FTMS without ECD. The remainder of the sequence was covered, with considerable redundancy, by abundant, easily detected peptides. Of the peptides in residues 22-104, every residue is accounted for by one or more N terminal cleavages except those below the mass range of the experiment or N terminal to a proline. This absence of cleavage N terminal to proline is predicted by the proposed mechanism for ECD fragmentation.

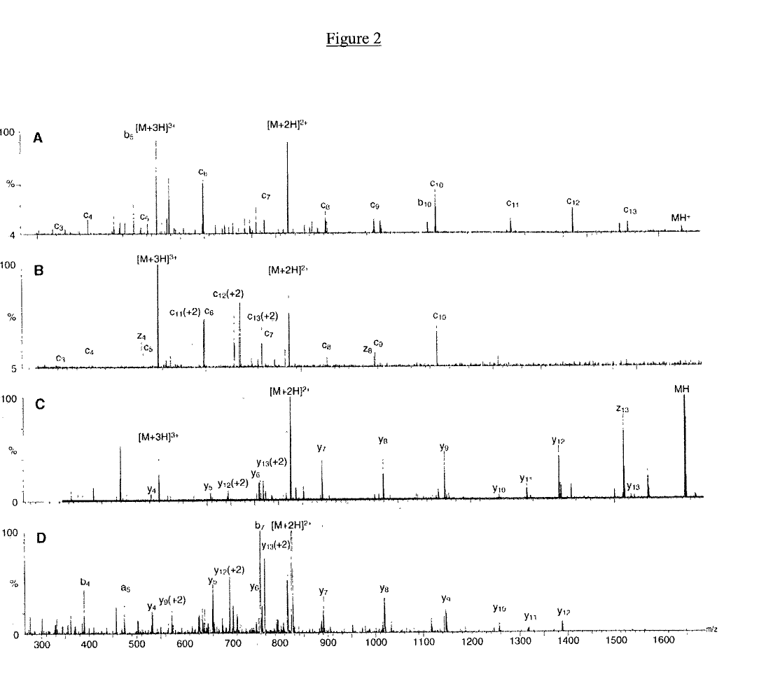

The spectra obtained from the peptic peptide of residues 81- 94 (IFAGIKKKTEREDL) illustrate features typical in all those acquired by μHPLC/ECD-FTMS. FIG. 2A shows the ECD fragmentation observed for this peptide. All possible c ions and most b ions are clearly seen. FIG. 2B shows this same peptide fragmented using μHPLC/ECD/FTMS with parent ion isolation of the [M+3H]³⁺ ion. This spectrum is similar and also contains all possible c ions. In this case c₁₁, c₁₂ and C₁₃ are present as doubly charged ions. This experiment also shows the utility of μHPLC/ECD-FTMS/MS in situations where true MS/MS information is required. For comparison the spectra observed by μHPLC/FTMS with in-source cone skimmer fragmentation is shown in FIG. 2C. The series y₆ to Y₁₂ is predominant. Only b₁₃²⁺ was prominent from the possible N terminal fragments. FIG. 2D shows this peptide subjected to LC/MS/MS using a triple quadrupole instrument. As expected this spectrum is very similar to the in source fragmentation; being dominated by a series of y fragment ions from y₄ to y₁₃. Thus no fragmentation is observed for the first 3 residues via in source or quadrupole collision cell fragmentation. The ECD fragmentation pattern is thus very different and in that sense provides complimentary information. The ECD spectrum is also complete and thus more informative for purposes of interpretation or confirmation of the sequence. The μHPLC/ECD-FTMS and μHPLC/ECD-FTMS/MS experiments were found to have sensitivity comparable to the triple quad MS/MS experiment, but less than conventional μHPLC/FTMS/MS.

TABLE 1
N-Terminal Fragmentation Observed in the Pepsin Digest of cytochrome c by μHPLC/ECD/FTMS. The cytochrome c sequence from residues 22-104 is
shown across the top of the Table. Individual observed peptic peptides are shown below relative to their location in the sequence below.
Those individual N terminal ECD fragments experimentally observed are indicated for each residue of each peptide. The three letter amino
acid code indicates that this residue could be verified by the mass difference between the molecular weight and the last c ion.

| Amino | 22 | 23 | 24 | 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 | 61 | 62 | 63 |
|---|---|---|---|---|---|---|---|---|---|---|---|---|---|---|---|---|---|---|---|---|---|---|---|---|---|---|---|---|---|---|---|---|---|---|---|---|---|---|---|---|---|---|
| Acid # | | | | | | | | | | | | | | | | | | | | | | | | | | | | | | | | | | | | | | | | | | |
| Se- | K | G | G | K | H | K | T | G | P | N | L | H | G | L | F | G | R | K | T | G | Q | A | P | G | F | T | Y | T | D | A | N | K | N | K | G | I | T | W | K | E | E | T |
| quence | | | | | | | | | | | | | | | | | | | | | | | | | | | | | | | | | | | | | | | | | | |
| 22-32 | K | G | G | K | H | K | T | G | P | N | L | | | | | | | | | | | | | | | | | | | | | | | | | | | | | | | |
| | | | . | b,c | b,c | b,c | c | . | c | b,c | Leu | | | | | | | | | | | | | | | | | | | | | | | | | | | | | | | |
| 22-36 | K | G | G | K | H | K | T | G | P | N | L | H | G | L | F | | | | | | | | | | | | | | | | | | | | | | | | | | | |
| | | | . | b | a,c | b,c | c | . | c | c | c | c | c | c | Phe | | | | | | | | | | | | | | | | | | | | | | | | | | | |
| 37-47 | | | | | | | | | | | | | | | | G | R | K | T | G | Q | A | P | G | F | T | | | | | | | | | | | | | | | | |
| | | | | | | | | | | | | | | | | | | b,c | c | c | c | b | c | c | b,c | Thr | | | | | | | | | | | | | | | | |
| 37-57 | | | | | | | | | | | | | | | | G | R | K | T | G | Q | A | P | G | F | T | Y | T | D | A | N | K | N | K | G | I | | | | | | |
| | | | | | | | | | | | | | | | | | | c | c | c | c | . | c | c | c | c | c | c | b,c | c | c | b,c | c | c | c | Ile | | | | | | |
| 47-64 | | | | | | | | | | | | | | | | | | | | | | | | | | T | Y | T | D | A | N | K | N | K | G | I | T | W | K | E | E | T |
| | | | | | | | | | | | | | | | | | | | | | | | | | | | | . | c | b,c | b,c | b,c | c | b,c | b,c | b,c | c | c | . | c | . | c |
| 48-64 | | | | | | | | | | | | | | | | | | | | | | | | | | | Y | T | D | A | N | K | N | K | G | I | T | W | K | E | E | T |
| | | | | | | | | | | | | | | | | | | | | | | | | | | | | | c | b,c | b,c | c | c | b,c | c | b,c | c | c | c | . | c | c |

| Amino | 66 | 66 | 67 | 68 | 69 | 70 | 71 | 72 | 73 | 74 | 75 | 76 | 77 | 78 | 79 | 80 | 81 | 82 | 83 | 84 | 85 | 86 | 87 | 88 | 89 | 90 | 91 | 92 | 93 | 94 | 95 | 96 | 97 | 98 | 99 | 100 101 102 103 | 104 |
|---|---|---|---|---|---|---|---|---|---|---|---|---|---|---|---|---|---|---|---|---|---|---|---|---|---|---|---|---|---|---|---|---|---|---|---|---|---|
| Acid # | | | | | | | | | | | | | | | | | | | | | | | | | | | | | | | | | | | | | |
| Se- | M | E | Y | L | E | N | P | K | K | Y | I | P | G | T | K | M | I | F | A | G | I | K | K | K | T | E | R | E | D | L | I | A | Y | L | K | K A T N | E |
| quence | | | | | | | | | | | | | | | | | | | | | | | | | | | | | | | | | | | | | |
| 65-80 | M | E | Y | L | E | N | P | K | K | Y | I | P | G | T | K | M | | | | | | | | | | | | | | | | | | | | | |
| | | | b,c | b,c | b,c | b | c | b,c | b,c | c | b | c | c | c | c | Met | | | | | | | | | | | | | | | | | | | | | |
| 65-82 | M | E | Y | L | E | N | P | K | K | Y | I | P | G | T | K | M | I | F | | | | | | | | | | | | | | | | | | | |
| | | | c | b | b,c | . | c | b,c | b,c | c | b | c | c | c | b,c | c | c | Phe | | | | | | | | | | | | | | | | | | | |
| 67-80 | | | Y | L | E | N | P | K | K | Y | I | P | G | T | K | M | | | | | | | | | | | | | | | | | | | | | |
| | | | | | b,c | b | c | b,c | b,c | c | b | c | c | c | b,c | Met | | | | | | | | | | | | | | | | | | | | | |
| 67-82 | | | Y | L | E | N | P | K | K | Y | I | P | G | T | K | M | I | F | | | | | | | | | | | | | | | | | | | |
| | | | | | b,c | b | c | b,c | b,c | a,b,c | b | c | c | c | b,c | c | c | Phe | | | | | | | | | | | | | | | | | | | |
| 68-80 | | | | L | E | N | P | K | K | Y | I | P | G | T | K | M | I | | | | | | | | | | | | | | | | | | | | |
| | | | | | | . | c | c | c | c | . | c | c | c | b,c | Met | | | | | | | | | | | | | | | | | | | | | |
| 68-82 | | | | L | E | N | P | K | K | Y | I | P | G | T | K | M | I | F | | | | | | | | | | | | | | | | | | | |
| | | | | | | b | c | b,c | b,c | a,b,c | b,c | c | c | c | b,c | c | c | Phe | | | | | | | | | | | | | | | | | | | |
| 81-94 | | | | | | | | | | | | | | | | | I | F | A | G | I | K | K | K | T | E | R | E | D | L | | | | | | | |
| | | | | | | | | | | | | | | | | | | | b | b,c | b,c | b,c | b,c | b,c | c | b,c | c | c | b,c | Leu | | | | | | | |
| 81-96 | | | | | | | | | | | | | | | | | I | F | A | G | I | K | K | K | T | E | R | E | D | L | I | A | | | | | |
| | | | | | | | | | | | | | | | | | | | b | b,c | a,b,c | b,c | b,c | b,c | c | b,c | c | c | c | . | c | Ala | | | | | |
| 83-94 | | | | | | | | | | | | | | | | | | | A | G | I | K | K | K | T | E | R | E | D | L | | | | | | | |
| | | | | | | | | | | | | | | | | | | | | | . | b,c | b,c | b,c | c | b,c | b,c | c | b,c | Leu | | | | | | | |
| 83-96 | | | | | | | | | | | | | | | | | | | A | G | I | K | K | K | T | E | R | E | D | L | I | A | | | | | |
| | | | | | | | | | | | | | | | | | | | | | . | b,c | b,c | b,c | c | b,c | c | c | b,c | b | c | Ala | | | | | |
| 95-104 | | | | | | | | | | | | | | | | | | | | | | | | | | | | | | | I | A | Y | L | K | K A T N | E |
| | | | | | | | | | | | | | | | | | | | | | | | | | | | | | | | | | b | b | b,c | b,c b,c b,c b,c | Glu |
| 97-104 | | | | | | | | | | | | | | | | | | | | | | | | | | | | | | | | | Y | L | K | K A T N | E |
| | | | | | | | | | | | | | | | | | | | | | | | | | | | | | | | | | | | c | b,c b,c b,c b,c | Glu |

The application of μHPLC/ECD-FTMS for the analysis of protein enzymatic digests is shown to produce spectra of high information content. These are well suited to the determination of unknown amino acid sequence or verification of sequence for quality control purposes. These are useful as a sole or primary analytical technique. The spectra are markedly different from spectra produced by other fragmentation techniques and thus can be used to complement those experiments. The previously reported advantages of ECD-FTMS for analysis of labile post-translational modifications could be obtained simultaneously. Pepsin is likely not the enzyme of choice for general applications. The most commonly employed enzyme, trypsin, has been favored in part because it yields peptides that produce superior fragmentation via CAD. It may evolve that other enzymatic cleavages are optimal for use with ECD, and the corresponding enzymes would therefore also be suitable for use in the present method.

It is anticipated that future developments of this novel technique will provide better sensitivity. It will also, hopefully, encourage efforts to commercialize enhancements in instrument control thus allowing the incorporation of data dependent parent isolation steps controlled in real time. This would permit the acquisition of very comprehensive fragmentation information, as demonstrated here, to be combined with the benefits of MS/MS for data interpretation.

### REFERENCES CITED

1) Katta, V., Chowdhury, S. K., and Chait, B. T. Anal. Chem. 1991; 63: 174.

2) Hunt, D. F. Yates III, J. R. Shabanowitz, J. Winston, S. and Hauer, C. R. PNAS 1986; 83: 6233.

3) Hess, D., Covey, T. R. Winz, R., Brownsey, R. W. and Abersold, R. Protein Science 1993; 2: 1342.

4) Covey, T. R., Huang, E. C. and Henion, J. D. Anal. Chem. 1991; 66: 1193.

5) Papayannopoulos, I. A. Mass Spectr. Rev. 1995; 14: 49.

6) Senko, M. W. Speir, J. P., and McLafferty, F. W. Anal. Chem. 1994; 66: 2809.

7) McLafferty, F. W., Horn, D. M., Breuker, K., Ge Y., Lewis, Cerda, B. Zubarev, and Carpenter, B. K. J. Am. Soc. Mass Sectrom. 2001; 12: 245.

8) Horn, D. M. Zubarev and Mclafferty, F. W. PNAS, 2000: 97: 103134.

9) Kruger, N. A. Zubarev, R. A., Carpenter, B. K. Kelleher, N. L. Horn, D. M. and McLafferty, F. W. Int. J. of Mass Spectrom. 1999; 182-183: 1.

10) Hakansson, K. Emmett, M. R. Hendrickson, C. L., and Marshall, A. G. Anal. Chem. 2001; 73: 3605.

11) Horn, D. M., Ge, Y., McLafferty, F. W. Anal. Chem. 2000; 72: 4778-4774.

12) Zubarev, R. A. Kelleher, N. L. McLafferty, F. W. J. Am Chem. Soc. 1998; 120: 3265.

13) Mirgorodskaya, E. Roepstorff and Zubarev, R. A., Anal Chem., 1999; 71: 4431.

14) Flora, J. W. and Muddiman, D. C., Anal Chem. 2001; 73: 3305.

15) Stensballe, A. Jensen, O. N., Olsen, J. V. Haselmann, K. F. and Zubarev, R. A. Rapid Commun. Mass Spectrom. 2000; 14: 1793.

16) Kruger, N. A. Zubarev, R. A. Horn, D. M. McLafferty, F. W. Int J. of Mass Spectrom. 1999; 185-187: 787.

17) Engen, John R., and Smith, David L. Methods in Molecular Biology-Mass Spectrometry of Proteins and Peptides 2000; 146: 95.

18) Dharmasiri, K., and Smith, D. L. Anal. Chem. 1996; 68: 2340.

19)Zhang, Z., and Smith, D. L. Protein Science 1993; 2: 522.

What is claimed is:

1. A process for analyzing a protein, comprising:
(a) digesting a protein with an enzyme to produce a protein digest;
(b) subjecting the protein digest produced in step (a) to liquid chromatography to separate the protein digest into components;
(c) ionizing the protein digest components produced in step (b) to produce multiply charged ions;
(d) trapping the ions produced in step (c) in an analysis cell of a fourier transform mass spectrometer;
(e) irradiating the trapped ions of step (d) with electrons to produce fragment ions; and
(f) obtaining mass spectral data with respect to the fragment ions produced in step (e)

2. A process according to claim 1, wherein the enzyme in step (a) is pepsin, trypsin, chymotrypsin or Endo Lys C.

3. A process according to claim 1, wherein the liquid chromatography in step (b) is reversed phase high pressure liquid chromatography.

4. A process according to claim 1, wherein the ionizing of step (c) is conducted using electrospray ionization.

5. A process according to claim 1, wherein the electrons of step (e) are produced by a metal filament.

6. A process according to claim 1, wherein ions are produced and isolated in step (c) using a mass spectrometer other than a fourier transform mass spectrometer, and these ions are then admitted into a fourier transform mass spectrometer for trapping in step (d).

7. A process according to claim 1, wherein there is no parent ion isolation during the process.

8. A process according to claim 1, wherein step (d) is followed by one or more parent ion isolation steps prior to the irradiation of the trapped ions in step (e).

9. A process according to claim 1, wherein the process is run continuously to obtain multiple spectra with respect to the fragment ions that are produced in step (e).

10. A process according to claim 1, wherein the protein digest components produced in step (b) are introduced directly into a mass spectrometer capable of performing the subsequent ionization step (c).

11. A process for analyzing a protein, comprising:
(a) digesting a protein with pepsin to produce a protein digest;
(b) subjecting the protein digest produced in step (a) to reversed phase high pressure liquid chromatography to separate the protein digest into components, and introducing the resulting protein digest components directly into a mass spectrometer capable of performing the subsequent ionization step (c);
(c) ionizing the protein digest components produced in step (b) using electrospray ionization to produce multiply charged ions;
(d) trapping the ions produced in step (c) in an analysis cell of a fourier transform mass spectrometer;
(e) irradiating the trapped ions of step (d) with electrons to produce fragment ions; and
(f) obtaining mass spectral data with respect to the fragment ions produced in step (e).

12. A process according to claim 11, wherein there is no parent ion isolation during the process.

13. A process according to claim 11, wherein step (d) is followed by one or more parent ion isolation steps prior to the irradiation of the trapped ions in step (e).

14. A process according to claim 11, wherein the process is run continuously to obtain multiple spectra with respect to the fragment ions that are produced in step (e).
